SEQUENCE LISTING (17 sequences)

SEQ ID NO: 1 - DNA, 19 bp, Artificial Sequence
  mouse NF-kB(p65) antisense oligo
gaaacagatcgtccatggt

SEQ ID NO: 2 - DNA, 19 bp, Artificial Sequence
  human NF-kB(p65) antisense oligo
ggaacagttcgtccatggc

SEQ ID NO: 3 - DNA, 19 bp, Artificial Sequence
  scrambled mouse NF-kB(p65) antisense oligo
agctgagtacgctatagac

SEQ ID NO: 4 - DNA, 1650 bp, Mus musculus
  misc_feature: Mouse complete NF-kB(p65) sequence as given by Gene bank accession number M61909
atggacgatctgtttcccctcatctttccctcagagccagcccaggcttctgggccttatgtggagatcatcgaacagccgaagcaacggggcatgcgattccgctataaatgcgaggggcgctcagcgggcagtattcctggcgagagaagcacagataccaccaagacacaccccaccatcaagatcaatggctacacaggaccaggaacagttcgaatctccctggtcaccaaggatccacctcaccggcctcatccacatgaacttgtggggaaggactgccgggatggctactatgaggctgacctctgcccagaccgcagtatccatagcttccagaacctggggatccagtgtgtgaagaagcgagacctggagcaagccattagccagcgaatccagaccaacaataacccctttcacgttcctatagaggagcagcgcggggactatgacttgaatgcagtgcgcctctgcttccaggtgacagtgcgggacccagcaggcaggcccctcctcctgacccctgtcctctcacatccgatttttgataaccgggcccccaacactgccgagctcaagatctgccgagtaaaccggaactctgggagctgcctcggtggggatgagatcttcttgctgtgcgacaaggtgcagaaagaagacattgaggtgtatttcacgggaccaggctgggaggcacgaggctccttttctcaagctgatgtgcatcggcaagtggccattgtgttccggactcctccgtacgccgaccccagcctccaggctcctgttcgagtctccatgcagctacggcggccttctgatcgcgagctcagtgagcccatggagttccagtacttgccagacacagatgatcgccaccggattgaagagaagcgcaaaaggacctatgagaccttcaagagtatcatgaagaagagtcctttcaatggaccaactgaaccccggcctccaacccggcgtattgctgtgcctacccgaaactcaacttctgtccccaagccagccccgcagccctacaccttcccagcatccctcagcaccatcaactttgatgagttttcccccatgctgttaccatcagggcagatctcaaaccaggccctggccttagcaccgtcctctgccccagtccttgcccagaccatggtcccttcctcagccatggtacctctggctcagcccccagctcctgccccagttctaaccccgggtcctccccagtccctgtctgcacctgttccaaagagcacccaggctggggaaggcacgctgtcggaagccctgctgcacctgcagtttgatgctgatgaagacttgggggccttgcttggcaacagcacagacccaggagtgttcacagacctggcatctgtggacaactcagagtttcagcagctcctgaaccagggtgtgtccatgtctcactccacagctgagcccatgctgatggagtaccctgaagctataactcgcctggtgacagggtcccagaggccccctgacccagctcccacacccctggggacctcggggcttcccaatggtctctccggagatgaagacttctcctccattgcggacatggacttctctgctcttttgagtcagatcagctcctaa

SEQ ID NO: 5 - DNA, 1656 bp, Homo sapiens
  misc_feature: Human complete NF-kB(p65) sequence as given by Gene bank accession number M62399
atggacgaactgttccccctcatcttcccggcagagccagcccaggcctctggcccctatgtggagatcattgagcagcccaagcagcggggcatgcgcttccgctacaagtgcgaggggcgctccgcgggcagcatcccaggcgagaggagcacagataccaccaagacccaccccaccatcaagatcaatggctacacaggaccagggacagtgcgcatctccctggtcaccaaggaccctcctcaccggcctcacccccacgagcttgtaggaaaggactgccgggatggcttctatgaggctgagctctgcccggaccgctgcatccacagtttccagaacctgggaatccagtgtgtgaagaagcgggacctggagcaggctatcagtcagcgcatccagaccaacaacaaccccttccaagttcctatagaagagcagcgtggggactacgacctgaatgctgtgcggctctgcttccaggtgacagtgcgggacccatcaggcaggcccctccgcctgccgcctgtccttcctcatcccatctttgacaatcgtgcccccaacactgccgagctcaagatctgccgagtgaaccgaaactctggcagctgcctcggtggggatgagatcttcctactgtgtgacaaggtgcagaaagaggacattgaggtgtatttcacgggaccaggctgggaggcccgaggctccttttcgcaagctgatgtgcaccgacaagtggccattgtgttccggacccctccctacgcagaccccagcctgcaggctcctgtgcgtgtctccatgcagctgcggcggccttccgaccgggagctcagtgagcccatggaattccagtacctgccagatacagacgatcgtcaccggattgaggagaaacgtaaaaggacatatgagaccttcaagagcatcatgaagaagagtcctttcagcggacccaccgacccccggcctccacctcgacgcattgctgtgccttcccgcagctcagcttctgtccccaagccagcaccccagccctatccctttacgtcatccctgagcaccatcaactatgatgagtttcccaccatggtgtttccttctgggcagatcagccaggcctcggccttggccccggcccctccccaagtcctgccccaggctccagcccctgcccctgctccagccatggtatcagctctggcccaggccccagcccctgtcccagtcctagccccaggccctcctcaggctgtggccccacctgcccccaagcccacccaggctggggaaggaacgctgtcagaggccctgctgcagctgcagtttgatgatgaagacctgggggccttgcttggcaacagcacagacccagctgtgttcacagacctggcatccgtcgacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctcctaa

SEQ ID NO: 6 - RNA, 21 nt, Homo sapiens
  misc_RNA (1)..(21): IDX 101
aaggaccuaugagaccuucaa

SEQ ID NO: 7 - RNA, 21 nt, Homo sapiens
  misc_RNA (1)..(21): IDX 105
aagaucaauggcuacacagga

SEQ ID NO: 8 - RNA, 21 nt, Homo sapiens
  misc_RNA (1)..(21): IDX 106
aacacugccgagcucaagauc

SEQ ID NO: 9 - RNA, 21 nt, Homo sapiens
  misc_RNA (1)..(21): IDX 107
gagucagaucagcuccuaagg

SEQ ID NO: 10 - DNA, 21 bp, Homo sapiens
  misc_RNA (1)..(21): IDX 121
uugaaggucucauaggucctt

SEQ ID NO: 11 - DNA, 21 bp, Homo sapiens
  misc_RNA (1)..(21): IDX 125
uccuguguagccauugauctt

SEQ ID NO: 12 - DNA, 21 bp, Homo sapiens
  misc_RNA (1)..(21): IDX 126
gaucuugagcucggcagugtt

SEQ ID NO: 13 - DNA, 21 bp, Homo sapiens
  misc_RNA (1)..(21): IDX 127
ccuuaggagcugaucugactt

SEQ ID NO: 14 - DNA, 21 bp, Homo sapiens
  misc_RNA (1)..(21): IDX 131
ggaccuaugagaccuucaatt

SEQ ID NO: 15 - DNA, 21 bp, Homo sapiens
  misc_RNA (1)..(21): IDX 135
gaucaauggcuacacaggatt

SEQ ID NO: 16 - DNA, 21 bp, Homo sapiens
  misc_RNA (1)..(21): IDX 136
cacugccgagcucaagauctt

SEQ ID NO: 17 - DNA, 21 bp, Homo sapiens
  misc_RNA (1)..(21): IDX 137
gucagaucagcuccuggaatt

(19) United States
(12) Patent Application Publication
(10) Pub. No.: US 2007/0298021 A1
(43) Pub. Date: Dec. 27, 2007

(54) Methods and Compositions for the Treatment or Prevention of Secondary Ischemic Injury

(75) Inventors: Oliver Von Stein, Spanga (SE); Sven Pettersson, Tullinge (SE); Sai Kiang Lim, Genome (SG)

Correspondence Address: DLA PIPER US LLP, 4365 EXECUTIVE DRIVE, SUITE 1100, SAN DIEGO, CA, 92121-2133, US

(73) Assignee: InDex Pharmaceuticals AB, Spanga (SE)

(21) Appl. No.: 10/589,842

(22) PCT Filed: Feb. 21, 2005
(86) PCT No.: PCT/SE05/00231

§ 371 Date: May 29, 2007

Related U.S. Application Data
(60) Provisional application No. 60/546,723, filed on Feb. 20, 2004.

(30) Foreign Application Priority Data
Feb. 20, 2004 (SE) 0400399.2

(51) Int. Cl. A61K 31/7088; A61K 31/727; A61K 38/43; A61P 9/00; A61P 9/10

(52) U.S. Cl. 424/94.1; 514/25; 514/44

(57) ABSTRACT

After a primary ischemic injury, which predominantly results in necrosis, there is a secondary injury in the neighbouring tissue, due at least to some extent to apoptosis. This secondary damage is usually not evident until several days after the initial ischemic event. The present invention provides methods of preventing, treating and/or alleviating secondary ischemic damage in a mammalian organ or tissue, comprising a step of administering an effective amount of an NF-κB inhibitor to said organ or tissue. Compositions for this purpose are also disclosed.

### FIELD OF THE INVENTION

The present invention relates to the treatment, prevention and/or alleviation of ischemic injury or damage in mammalian organs or tissue, in particular secondary ischemic damage. More specifically, the invention relates to methods of treatment, as well as the use of inhibitors of NF-κB p65 in the manufacture of pharmaceutical preparations for the treatment, prevention and/or alleviation of secondary ischemic damage, occurring in an organ or tissue inter alia following vascular occlusion, as in myocardial infarction, surgical intervention, trauma, transplantation etc.

### BACKGROUND OF THE INVENTION

The following is a brief description of the physiological role of NF-κB. The discussion is not meant to be complete and is provided only for understanding the invention that follows. This background is not an admission that any of the research or references described herein is prior art to the claimed invention.

Transcription factors represent a group of molecules within the cell that function to connect the pathway from extracellular signal to intracellular response. Immediately after an environmental stimulus, these proteins, which reside predominantly in the cytosol, are translocated to the nucleus where they bind to DNA and activate the transcription of their target genes. One such group of transcription factors is the Rel/NF-κB family. These transcriptional activators are found in many tissues and are localized in the cytosol as hetero- or homodimers with other members of the family and remain inactive due to the association of the dimer with an inhibitory protein. Rel/NF-κB proteins have been shown to be involved in a diverse set of signalling pathways involving stress, apoptosis, cancer, growth, infection, and inflammation.

NF-κB is a multi-protein complex, which can rapidly induce the synthesis of defence and signalling proteins upon exposure of cells to a wide variety of mostly pathogenic agents. Three protein subunits, IκB, p50 and p65, control the biological functions of NF-κB. IκB is a 35-43 kDa subunit, which inhibits the DNA-binding of NF-κB and serves to retain NF-κB in an inducible form in the cytoplasm of unstimulated cells. It does so by forming a tertiary complex with p50/p65, of which it binds to p65. Upon stimulation of cells, IκB dissociates from the inactive complex with p50/p65. The released p50/p65 complex can then migrate into the nucleus and potently transactivate genes, p50 and p65 being the two DNA binding domains of NF-κB. The combination of p50 and p65 DNA binding subunits in NF-κB extends the repertoire of binding motifs recognized with high affinity.

In addition to the complex with p50, p65 can also associate with unprocessed p105, another Rel family protein. The p65/p105 heterodimer can be activated by processing of p105. Thus, NF-κB's p65 subunit can form homo- and heterodimers, once the dimer dissociates from the inhibitor molecule it translocates to the nucleus and binds to the less conserved half site of κB motifs. In the nucleus it affects the transcription of several target genes, including NF-κB family members and those of the inhibitors.

In addition to its role in normal cellular processes, manifestations of altered NF-κB p65 subunit regulation appear in disease states, making p65 an interesting target for therapeutic intervention. NF-κB has been implicated as a key factor in inflammatory conditions, and Pettersson et al. (WO 97/47325) showed that local administration of p65 antisense oligonucleotides was effective in down regulating the expression of cytokines in inflammatory conditions, such as Crohn's disease or rheumatoid arthritis.

To date, strategies aimed at inhibiting and/or investigating NF-κB p65 subunit function have involved the use of small molecular inhibitors, antibodies, decoys (i.e. a compound which specifically antagonizes the nucleic acid domain to which NF-kappa B is bound), dominant-negative forms of NF-κB p65 subunit, ribosome inhibition, siRNA constructs, antisense technology and gene knock-outs in mice.

Antisense molecules targeted to NF-κB p65 subunit are known. Antisense oligonucleotides to the corresponding section of the NF-κB p65 subunit gene in mice have also been used to investigate NF-κB p65 subunit function.

For example, Nerenberg and Kitajima (U.S. Pat. No. 6,498,147, WO 95/35032) disclose antisense oligonucleotides, which hybridise with NF-κB mRNA and methods of using these oligonucleotides.

Narayanan and Rosen (U.S. Pat. No. 5,591,840) teaches oligodeoxynucleotides which are capable of hybridising to genes which encode NF-kB. The oligodeoxyribonucleotides are antisense to NF-kB genes and when hybridised prevent the production of NF kappa B transcription factor by the NF kappa B genes, and thereby provide a means for preventing cellular adhesion.

Further, Bennett et al. (U.S. Pat. No. 6,069,008) discloses antisense compounds, compositions and methods for modulating the expression of NF-kB p65 subunit. The compositions comprise antisense compounds, particularly antisense oligonucleotides, targeted to nucleic acids encoding NF-kB p65 subunit. Methods of using these compounds for modulation of NF-kB p65 subunit expression and for treatment of diseases associated with expression of NF-kB p65 subunit are provided.

In some instances, NF-κB p65 has been linked to genes involved in the ischemic response. Ischemia, i.e. a decreased supply of oxygenated blood to a body organ of part, often marked by pain and organ dysfunction, and which results in cellular damage and death when prolonged. The damage or injury is also known as infarction.

Cellular damage and death occurs through two routes, necrosis and apoptosis. Necrosis stems from an external injury, which produces cellular membrane destruction, swelling and lysis. Apoptosis is a process of endogenously-mediated cellular suicide affected by activation of a series of aspartate-specific proteases called caspases and endonucleases, resulting in proteolytic destruction of cellular proteins and chromosomal elements. In myocardial infarction, both processes contribute to myocardial muscle injury and destruction. Overt necrosis predominates in the central zone of infarcted myocardium, and apoptosis occurs in the border zones of histologically infarcted myocardium.

Generally, apoptosis is controlled at two distinct levels. First, cells have unique sensors, termed death receptors, on their membrane surface. Death receptors detect the presence of extracellular death signals and, in response, ignite the cell's intrinsic apoptosis machinery. Second, activation of caspases and subsequent apoptosis are initiated by events that disturb mitochondria. Either disruption of electron transport and aerobic oxidative phosphorylation or opening of pores in the outer mitochondrial membrane allows cytochrome c to leak out of the mitochondria. Through further interactions, cytochrome c helps activate the caspase cascade and initiation of cellular apoptosis.

Morishita et al. (WO 96/35430, U.S. Pat. No. 6,262,033) have shown that the administration of a decoy, i.e. a compound which specifically antagonizes the nucleic acid domain to which NF-κB is bound, is effective in the treatment and prevention of diseases caused by the transcriptional regulatory factor NF-κB, such as ischemic diseases, inflammatory diseases, autoimmune diseases, cancer metastasis and invasion, and cachexia. Morishita et al. are however silent on the issue of secondary ischemic damage, and the role of NF-κB in the development of ischemic damage in neighbouring tissue. Notably, the rats used in the experiments of Morishita et al. were killed already 24 hours following reperfusion. Secondary ischemic damage could therefore not be detected.

Brand et al. (U.S. Pat. No. 6,271,199) concerns the treatment of ischemia by administering proteasome inhibitors, ubiquitin pathway inhibitors, agents that interfere with the activation of NF-κB via the ubiquitin proteasome pathway, or mixtures thereof.

Kennedy et al. (U.S. Pat. No. 6,489,311) compared heparin and an O-desulfated nonanticoagulant heparin with greatly reduced anti-complement activity. Given at the time of coronary artery reperfusion in a canine model of myocardial infarction, both heparin and O-desulfated heparin equally reduced neutrophil adherence to ischemic-reperfused coronary artery endothelium, influx of neutrophils into ischemic-reperfused myocardium, myocardial necrosis and release of creatine kinase into plasma. Heparin and O-desulfated heparin also prevented dysfunction of endothelial-dependent coronary relaxation following ischemic injury. In addition, heparin and O-desulfated heparin inhibited translocation of the transcription factor NF-kB from cytoplasm to the nucleus in human endothelial cells and decreased NF-kB DNA binding in human endothelium and ischemic-reperfused rat myocardium. Thus, Kennedy et al. found that heparin and non-anticoagulant heparin decrease ischemia-reperfusion injury by disrupting multiple levels of the inflammatory cascade, including the novel observation that heparins inhibit activation of the pro-inflammatory transcription factor NF-kB.

Huang et al. in Journal of Cerebral Blood Flow and Metabolism, 21:163-173, 2, 2001 have shown that overexpression of human copper/zinc-superoxide dismutase (SOD1) reduced ischemia-induced NF-kB DNA binding activity, as well as NF-kB protein levels in the ischemic brain. The article focuses on the role of reactive oxygen species (ROS) and its influence on transcription factors like NF-kB. It was already known that NF-kB responds to oxidative stress induced by ROS. The animal study performed by Huang et al. showed that the overexpression of SOD1 attenuated NF-kB activation after transient focal cerebral ischemia. This cannot however be seen as a direct approach to regulating NF-kB p65.

In WO 03/041640, Rosenzweig and Ahn disclose the use of IkB kinase-beta inhibitors fro treating ischemic reperfusion injury. A dominant negative IKK-beta mutant was shown to inhibit NF-kB activation in vivo.

In WO 03/070918, McSwiggen et al. present a siRNA approach to inhibiting gene expression, however without reference to the prevention of secondary ischemic damage.

US 2001/0002391 A1, by Brand et al, concerns the treatment of ischemia by administering proteasome inhibitors, ubiquitin pathway inhibitors, agents that interfere with the activation of NF-kB via the ubiquitin proteasome pathway, or mixtures thereof.

In summary, ischemia and in particular myocardial infarct has been extensively studied, but significant information on the progression of the infarct is still missing, as is more knowledge of preventatives or therapeutics. It is taught in the art that the magnitude of ischemic injury depends upon the degree and duration of the ischemic event, such as a coronary embolism. Therefore the previous focus tends to be related to minimizing or preventing the ischemic event. Some researchers have considered how to prevent or reduce the immediate consequences of ischemia. But there remains a need in the art to further define the subsequent stages of the ischemic process and direct treatments to stages heretofore un-investigated. In view of the foregoing, it is readily apparent that there is a need for further investigation of apoptosis and secondary injury ischemic events, such as in myocardial infraction, surgical intervention, trauma, transplantation, to mention a few examples, as well as potential methods and compositions for the treatment, prevention and/or prevention of such injury.

### SUMMARY OF THE INVENTION

The present inventors make available a method of preventing, reducing the extent and/or the severity of secondary ischemic damage in a mammalian organ or tissue, comprising a step of administering an effective amount of an NF-κB p65 inhibitor to said organ or tissue. The inventors also make available a method of preventing, reducing the extent and/or the severity of secondary myocardial infarct in a mammalian heart, comprising a step of administering an effective amount of an NF-κB p65 inhibitor to said heart.

The invention also discloses the use of an NF-κB p65 inhibitor for the manufacture of a pharmaceutical composition for preventing, reducing the extent and/or the severity of secondary ischemic damage in a mammalian organ or tissue; as well as for the manufacture of a pharmaceutical composition for protecting tissue adjacent to area of myocardial infarct in a mammal; or for the manufacture of a pharmaceutical composition for preventing, reducing the extent and/or the severity of reperfusion ischemia in a mammalian organ or tissue.

The invention also encompasses compositions and sub-sets of compositions e.g. in the form of a kit, for the simultaneous or consecutive administration of one or more NF-κB p65 inhibitors, and optionally one or more pharmacological agents for any of the disclosed therapeutic or prophylactic purposes.

Further embodiments of the invention are set out in the attached independent and dependent claims, incorporated herein by reference.

The sequences referred to in the description, examples and claims are as disclosed in the attached Sequence Listing, prepared using the PatentIn 3.1 software. SEQ ID NO. 1 is a mouse NF-κB (p65) antisense oligonucleotide; SEQ ID NO. 2 is a human NF-κB (p65) antisense oligonucleotide; SEQ ID NO. 3 is a scrambled mouse NF-κB (p65) antisense oligonucleotide used as control; SEQ ID NO. 4 is the murine NF-κB (p65) sequence (Gene Bank, M61909); and SEQ ID NO. 5 is the human NF-κB (p65) sequence (Gene Bank, M62399). The siRNA target sequences 6-9, and the corresponding lower and upper strands, sequences 10-13, and 14-17, respectively, are only shown in Table 1.

### BRIEF DESCRIPTION OF THE DRAWINGS

The invention will be described in closer detail in the following description, examples, and attached drawings, in which

FIG. 1 is a reproduction of a photography showing two mouse hearts, with the location of two dissection cuts shown as transverse lines (1st cut; 2nd cut);

FIGS. 2A, 2B, and 2C are reproductions of microscope photographs each showing a heart section, the section being taken at the 1st dissection cut from control, scrambled oligonucleotide and antisense oligonucleotide mice, respectively; and

FIG. 3 is a graph, schematically representing histology data. In the figure, solid black bars indicate values for saline control animals, striped bars represent data from scrambled oligonucleotide control animals, and checked bars indicate values from animals receiving the antisense NF-κB p65 oligonucleotide. Sections taken at the 1st cut, as well as the 2nd cut (results not shown) have been compared for each animal.

FIG. 4 shows an immuno blot analysis of p65 in human HeLa cells transfected (Gene Silencer) with 150 pmol of siRNA compound IDX0131. After transfection the cells were treated with 50 ng/mL 12-0-tetradecanoylphorbol-13-acetate (TPA) for 24 or 48 hr, as indicated, and harvested 50 ug of WCE was loaded in each lane. Equal loading was confirmed by staining the membrane with Ponseau S.

### DESCRIPTION

The invention relates to the treatment of ischemia in a way that prevents, treats and/or alleviates the severity of damage caused by an ischemic event in secondarily affected tissues. The present inventors have discovered that blocking NF-κB p65 subunit function reduces the secondary effects of ischemia, such as reducing secondary infarct size following vascular occlusion as well as reducing the post-ischemic thinning of the heart muscle wall. The invention can be practiced by administering an NF-κB inhibitor, e.g. an antisense NF-κB p65 oligonucleotide.

It has now been unexpectedly discovered that treatment and prevention of a primary physiological response to ischemia in an organ does not sufficiently address all damage caused by ischemia. Instead, after the primary injury, which predominantly results in necrosis, there is a secondary injury in the neighbouring tissue, due at least to some extent to apoptosis. This secondary damage is usually not evident until several days after the initial ischemic event.

It has unexpectedly been revealed through the present invention that the secondary ischemic injury can be reduced in severity through the use of an NF-κB p65 inhibitor, such as an antisense NF-κB p65 subunit oligonucleotide. These inhibitors can beneficially prevent, reduce the size, or lessen the severity of secondary infarction following vascular occlusions such as occur e.g. during a heart attack. Inhibitors of NF-κB p65 subunit activation can provide effective therapy for these conditions. Thus, the invention provides surprisingly effective methods for treating secondary ischemic damage due to initial ischemia, particularly secondary myocardial infarcts, secondary ischemic brain injury, secondary reperfusion injury in transplanted organs or tissue, in angioplasty and in the wake of surgical intervention or trauma, etc.

For convenience, certain terms employed in the specification, examples, and appended claims are collected here.

The terms “secondary ischemic damage” and “secondary myocardial infarct” refer to a condition or disease state that follow ischemia. The ischemic incident produces a primary ischemic damage that is distinct both in time and physical location from the secondary damage described herein.

The term “NF-κB inhibitor” encompasses all compounds and methods capable of modulating, preferably inhibiting, the activation of NF-κB and in particular the expression of the NF-κB p65 subunit. Examples of NF-κB inhibitors and inhibition strategies include, but are not limited to: small molecular inhibitors; an antisense NF-κB p65 subunit oligonucleotide; a dominant-negative form of the NF-κB p65 subunit; a decoy; ribosome inhibition; enzymatic RNA against NF-κB; and siRNA.

Antisense compounds are commonly used as research reagents and diagnostics. For example, antisense oligonucleotides, which are able to inhibit gene expression with exquisite specificity, are often used by those of ordinary skill to elucidate the function of particular genes. The specificity and sensitivity of antisense is also harnessed by those of skill in the art for therapeutic uses. Antisense oligonucleotides have been employed as therapeutic moieties in the treatment of disease states in animals and man. Antisense oligonucleotides have been safely and effectively administered to humans and numerous clinical trials are presently underway. It is thus established that oligonucleotides can be useful therapeutic modalities that can be configured to be useful in treatment regimes for treatment of cells, tissues and animals, especially humans.

In antisense therapy, generally, the oligonucleotide employed will have a sequence that is complementary to the sequence of the target RNA. However, absolute complementarity is not required; in general, any oligonucleotide having sufficient complementarity to form a stable duplex with the target RNA so that translation of the RNA is inhibited is considered to be suitable. Since stable duplex formation depends on the sequence and the length of the hybridising oligonucleotide and the degree of complementarity between the antisense oligonucleotide and the target sequence, the system can tolerate less complementarity when longer oligonucleotides are used. However, it is presently believed that oligonucleotides of about 8 to about 40 bases in length, preferably of about 8 to about 30 bases in length, and more preferably of about 8 to about 20 bases in length, and having a sufficient complementarity to for a duplex having a melting temperature of greater than about 40° C. under physiologic conditions are particularly well suited for practice of the invention. Accordingly, such oligonucleotides are preferred. Another variable that may affect practice of the invention is the region of the target RNA to which the selected oligonucleotide is designed to hybridise. Although oligonucleotides capable of stably hybridising with any region of the RNA may be suitable for practice of the invention, oligonucleotides complementary to a region including the translation initiation nucleic acid sequence of the NF-κB subunit are particularly effective. The antisense oligonucleotide is considered effective as long as the translation of the mRNA to which the oligonucleotide is complementary is inhibited.

In the context of this invention, the term “oligonucleotide” refers to an oligomer or polymer of ribonucleic acid (RNA) or deoxyribonucleic acid (DNA) or mimetics thereof. This term includes oligonucleotides composed of naturally occurring nucleobases, sugars and covalent internucleoside (backbone) linkages as well as oligonucleotides having non-naturally-occurring portions which function similarly. Such modified or substituted oligonucleotides are often preferred over native forms because of desirable properties such as, for example, enhanced cellular uptake, enhanced affinity for nucleic acid target and increased stability in the presence of nucleases. An oligonucleotide is usually comprised of more than three (3), and typically more than ten (10) and up to one hundred (100) or more deoxyribonucleotides or ribonucelotides, although preferably between about eight (8) and about forty (40), most preferably between about eight (8) and about twenty (20). The exact size will depend on many factors, which in turn depends on the ultimate function or use of the oligonucleotide. The oligonucleotide may be generated in any manner, including chemical synthesis, DNA replication, reverse transcription, or a combination thereof.

An “antisense oligonucleotide” or “antisense agent” refers to a molecule, which includes a sequence of purine and pyrimidine heterocyclic bases, supported by a backbone, which are effective to hydrogen-bond to corresponding, contiguous bases in a target nucleic acid sequence. In the present invention, the base sequence is complementary (antisense) to portions of p65 mRNA.

Various oligonucleotide analogs known in the art present advantages over “natural” polynucleotides in areas such as stability, particularly nuclease resistance, reduction of non-specific binding, and bioavailability (i.e. access to cells). The structures may be modified at the backbone, the sugar moiety, or the bases themselves. Examples known in the art include, but are not limited to phosphorothioate, morpholino oligonucleotides, peptide nucleic acids, 2′-O-allyl or 2′-O-alkyl modified oligonucleotides, or N3′->P5′ phosphoramidate oligonucleotides (See, e.g. U.S. Pat. No. 6,365,577)

“Targeting” an antisense compound to a particular nucleic acid, in the context of this invention, is a multi step process. The process usually begins with the identification of a nucleic acid sequence whose function is to be modulated. This may be, for example, a cellular gene (or mRNA transcribed from the gene) whose expression is associated with a particular disorder or disease state. In the present invention, the targets are nucleic acid molecules encoding NF-κB p65 subunit. The targeting process also includes determination of a site or sites within the gene for the antisense interaction to occur such that the desired effect, e.g., modulation of expression of the protein, will result.

As used herein, the terms “target nucleic acid” and “nucleic acid encoding NF-κB p65 subunit” encompass DNA encoding NF-κB p65 subunit, RNA (including pre-mRNA and mRNA) transcribed from such DNA, and also cDNA derived from such RNA. The specific hybridisation of an oligomeric compound with its target nucleic acid interferes with the normal function of the nucleic acid. The functions of DNA to be interfered with include replication and transcription. The functions of RNA to be interfered with include all vital functions such as, for example, translocation of the RNA to the site of protein translation, translation of protein from the RNA, splicing of the RNA to yield one or more mRNA species, and catalytic activity which may be engaged in or facilitated by the RNA.

Numerous references disclose in detail antisense techniques, for example, Jarad et al., Nucleic acid-based techniques for post-transcriptional regulation of molecular targets, Curr Opin Nephrol Hypertens. 2003 July; 12(4):415-21.

In the context of this invention, “hybridisation” means hydrogen bonding, which may be Watson-Crick, Hoogsteen or reversed Hoogsteen hydrogen bonding, between complementary nucleoside or nucleotide bases. For example, adenine and thymine are complementary nucleobases, which pair through the formation of hydrogen bonds. “Complementary,” as used herein, refers to the capacity for precise pairing between two nucleotides. For example, if a nucleotide at a certain position of an oligonucleotide is capable of hydrogen bonding with a nucleotide at the same position of a DNA or RNA molecule, then the oligonucleotide and the DNA or RNA are considered to be complementary to each other at that position. The oligonucleotide and the DNA or RNA are complementary to each other when a sufficient number of corresponding positions in each molecule are occupied by nucleotides which can hydrogen bond with each other. Thus, “specifically hybridisable” and “complementary” are terms which are used to indicate a sufficient degree of complementarity or precise pairing such that stable and specific binding occurs between the oligonucleotide and the DNA or RNA target. It is understood in the art that the sequence of an antisense compound need not be 100% complementary to that of its target nucleic acid to be specifically hybridisable. An antisense compound is specifically hybridisable when binding of the compound to the target DNA or RNA molecule interferes with the normal function of the target DNA or RNA to cause a loss of utility, and there is a sufficient degree of complementarity to avoid non-specific binding of the antisense compound to non-target sequences under conditions in which specific binding is desired, i.e., under physiological conditions in the case of in vivo assays or therapeutic treatment, and in the case of in vitro assays, under conditions in which the assays are performed.

As used herein the term “animal” refers to mammals, preferably mammals such as livestock or humans. Likewise, a “patient” or “subject” to be treated by the subject method can mean either a human or non-human animal.

The term “isolated” as also used herein with respect to nucleic acids, such as DNA or RNA, refers to molecules separated from other DNAs, or RNAs, respectively, which are present in the natural source of the macromolecule. The term isolated as used herein also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments, which are not naturally occurring as fragments and would not be found in the natural state.

Two DNA or polypeptide sequences are “substantially homologous” when at least about 75% (preferably at least about 80%, and most preferably at least about 90 or 95%) of the nucleotides match over the defined length of the DNA sequences. Sequences that are substantially homologous can be identified by comparing the sequences using standard software available in sequence data banks, or in a Southern hybridisation experiment under, for example, stringent conditions as defined for that particular system. Defining appropriate hybridisation conditions is within the skill of the art.

The phrase “pharmaceutically acceptable” refers to molecular entities and compositions that are physiologically tolerable and do not typically produce an allergic or similar untoward reaction, such as gastric upset, dizziness and the like, when administered to a patient.

The phrase “therapeutically effective amount” is used herein to mean an amount sufficient to suppress to some beneficial degree, preferably to reduce by at least about 30 percent, more preferably by at least 50 percent, most preferably by at least 90 percent, secondary ischemic damage. In the experimental part of this application, this is shown as a reduction of cavity size, reduction of wall thinning, and by increased size of underlying heart tissue as compared to untreated controls.

“Treatment” shall mean preventing or lessening secondary ischemic injury, including the prevention of infarction spread or reduction in size or lessening in severity of secondary infarct, including without limitation to infarct after vascular occlusion. Any amelioration of any symptom of the secondary infarct pursuant to treatment using any agent that interferes with activation of NF-κB p65 subunit is within the scope of the invention.

While antisense NF-κB p65 oligonucleotides are a preferred form of antisense compound, the present invention comprehends other oligomeric antisense compounds, including but not limited to oligonucleotide mimetics. The antisense compounds in accordance with this aspect of the invention preferably comprise from about 8 to about 40 nucleobases (i.e. from about 8 to about 40 linked nucleosides, preferably from about 8 to about 30, more preferably from about 8 to about 20 nucleobases). As is known in the art, a nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric compound. In turn the respective ends of this linear polymeric structure can be further joined to form a circular structure; however, open linear structures are generally preferred. Within the oligonucleotide structure, the phosphate groups are commonly referred to as forming the internucleoside backbone of the oligonucleotide. The normal linkage or backbone of RNA and DNA is a 3′ to 5′ phosphodiester linkage.

In other preferred oligonucleotide mimetics, both the sugar and the internucleoside linkage, i.e., the backbone, of the nucleotide units are replaced with novel groups. The base units are maintained for hybridisation with an appropriate nucleic acid target compound. One such oligomeric compound, an oligonucleotide mimetic that has been shown to have excellent hybridisation properties, is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. PNA compounds are taught in, inter alia, Braasch et al., Efficient and isoform-selective inhibition of cellular gene expression by Peptide nucleic acids, Biochemistry. 2004 Feb. 24; 43(7):1921-7.

Another approach is the use of locked nucleic acids (LNA), which have been successfully evaluated for antisense activity. At least two different diastereoisomers of LNA have been studied: the beta-D-LNA and the alpha-L-LNA (abbreviated as β-D-LNA and α-L-LNA). There are indications that the best antisense activity with 16 mer gapmers containing β-D-LNA (oligonucleotides containing consecutive segments of LNA and DNA with a central DNA stretch flanked by two LNA segments, LNA-DNA-LNA) is found with gap sizes between 7 and 10 nt. The optimal gap size is motif-dependent, and requires the right balance between gap size and affinity. Compared to β-D-LNA, α-L-LNA shows superior stability against a 3′-exonuclease. The design possibilities of α-L-LNA have been explored for different gapmers and other designs, collectively called chimeras. Published results indicate that α-L-LNA is a powerful and versatile nucleotide analogue for designing potent antisense oligonucleotides. See for example Wengel, et al., LNA and alpha-L-LNA: towards therapeutic applications, Nucleosides Nucleotides Nucleic Acids. 2003 May-August; 2(5-8):601-4, and Frieden et al., Expanding the design horizon of antisense oligonucleotides with alpha-L-LNA, Nucleic Acids Res. 2003 Nov. 1; 31 (21): 6365-6372.

Another means to interfere with gene expression contemplated by the present invention is to employ small interfering RNA (also known as short interfering RNA, or simply siRNA). A siRNA construct comprises a sense and antisense strand of RNA corresponding to a region of the gene of interest. Target sequences, and the corresponding upper and lower strands of preferred siRNA compounds or constructs are shown in Table 1.

TABLE 1

NF-kappa-B target and siRNA compound
sequences (5′-3′)

| | | |
|---|---|---|
| SEQ | | Applicant's |
| ID | | reference |
| NO | Sequence | no. |

Target Sequence

| | | |
|---|---|---|
| 6 | AAGGACCUAUGAGACCUUCAA | IDX 101 |
| 7 | AAGAUCAAUGGCUACACAGGA | IDX 105 |
| 8 | AACACUGCCGAGCUCAAGAUC | IDX 106 |
| 9 | GAGUCAGAUCAGCUCCUAAGG | IDX 107 |

Lower Sequence

| | | |
|---|---|---|
| 10 | UUGAAGGUCUCAUAGGUCC(D)TT* | IDX 121 |
| 11 | UCCUGUGUAGCCAUUGAUC(D)TT | IDX 125 |
| 12 | GAUCUUGAGCUCGGCAGUG(D)TT | IDX 126 |
| 13 | CCUUAGGAGCUGAUCUGAC(D)TT | IDX 127 |

Upper Sequence

| | | |
|---|---|---|
| 14 | GGACCUAUGAGACCUUCAA(D)TT* | IDX 131 |
| 15 | GAUCAAUGGCUACACAGGA(D)TT | IDX 135 |
| 16 | CACUGCCGAGCUCAAGAUC(D)TT | IDX 136 |
| 17 | GUCAGAUCAGCUCCUGGAA(D)TT | IDX 137 |

Note:
An asterisk (*) denotes that siRNA compound composed of antisense stand SEQ ID NO 5 annealed with sense strand SEQ ID NO 9 that was used in the animal proof of concept studies.

Compounds synthesized as RNA oligonucleotides with the last two nucleotides from the 3′ end are DNA, denoted as (D).

The 3′-ends of the Upper sequence and the Lower sequence of the siRNA construct can include an overhanging sequence, for example 1, 2, 3, or 4 nucleotides in length, preferably 2 nucleotides in length, wherein the overhanging sequence of the lower sequence is optionally complementary to a portion of the target sequence. The upper sequence is also referred to as the sense strand, whereas the lower sequence is also referred to as the antisense strand.

A siRNA molecule consists of approximately 19 nucleotides plus an overhang of approximately 2 nucleotides at the 3′ end. Some preferred methods include the use of siRNA constructs of between 19-23 nucleotides plus 3′ overhang. The siRNA is introduced to the cell or cells of interest through known methods. Following introduction, the cell or cells destroy ssRNA having the same sequence. This results in a reduction or prevention in translation of a targeted gene and a corresponding reduction or prevention in protein production. For information on siRNA, see e.g. Zender et al., SiRNA based strategies for inhibition of apoptotic pathways in vivo—analytical and therapeutic implications, Apoptosis. 2004 January; 9(1):51-4.

Modified oligonucleotides may also contain one or more substituted sugar moieties as known in the art. Oligonucleotides may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases.

Another modification of the oligonucleotides of the invention involves chemically linking to the oligonucleotide one or more moieties or conjugates, which enhance the activity, cellular distribution or cellular uptake of the oligonucleotide.

It is not necessary for all positions in a given compound to be uniformly modified, and in fact more than one of the aforementioned modifications may be incorporated in a single compound or even at a single nucleoside within an oligonucleotide. The present invention also includes antisense compounds, which are chimeric compounds. “Chimeric” antisense compounds or “chimeras,” in the context of this invention, are antisense compounds, particularly oligonucleotides, which contain two or more chemically distinct regions, each made up of at least one monomer unit, i.e., a nucleotide in the case of an oligonucleotide compound. These oligonucleotides typically contain at least one region wherein the oligonucleotide is modified so as to confer upon the oligonucleotide increased resistance to nuclease degradation, increased cellular uptake, and/or increased binding affinity for the target nucleic acid. An additional region of the oligonucleotide may serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of oligonucleotide inhibition of gene expression. Consequently, comparable results can often be obtained with shorter oligonucleotides when chimeric oligonucleotides are used, compared to phosphorothioate deoxyoligonucleotides hybridising to the same target region. Cleavage of the RNA target can be routinely detected by gel electrophoresis and, if necessary, associated nucleic acid hybridisation techniques known in the art.

Chimeric antisense compounds of the invention may be formed as composite structures of two or more oligonucleotides, modified oligonucleotides, oligonucleosides and/or oligonucleotide mimetics as described above. Such compounds have also been referred to in the art as hybrids or gapmers. References teaching the preparation of such hybrid structures include, but are not limited to: Grunweller et al., Comparison of different antisense strategies in mammalian cells using locked nucleic acids, 2′-O-methyl RNA, phosphorothioates and small interfering RNA, Nucleic Acids Res. 2003 Jun. 15; 31 (12): 3185-3193.

The compounds of the invention may also be admixed, encapsulated, conjugated or otherwise associated with other molecules, molecule structures or mixtures of compounds, as for example, liposomes, receptor targeted molecules, oral, rectal, topical or other formulations, for assisting in uptake, distribution and/or absorption. Vehicles or compositions for delivery are well known to persons skilled in the art and can be applied to the delivery of the compound of the present invention, with or without minor modifications, without involving an inventive effort.

The NF-κB p65 inhibitor compounds of the invention encompass any pharmaceutically acceptable salts, esters, or salts of such esters, or any other compound which, upon administration to an animal including a human, is capable of providing (directly or indirectly) the biologically active metabolite or residue thereof. Accordingly, for example, the disclosure is also drawn to prodrugs and pharmaceutically acceptable salts of the compounds of the invention, pharmaceutically acceptable salts of such prodrugs, and other bioequivalents.

According to one embodiment of the invention, the NF-κB inhibitor compound or compounds is/are administered substantially simultaneously, before or after the administration of at least one second agent selected from the group consisting of anticoagulants and anti-thrombotics, such as nut not limited to vitamin K-antagonists, heparin and heparinoid agents, platelet aggregation inhibitors and the like. Likewise, the NF-κB inhibitor compound may comprise a pharmaceutically effective and physiologically tolerated amount of at least one second agent selected from the group consisting of anticoagulants and anti-thrombotics, such as but not limited to vitamin K-antagonists, heparin and heparinoid agents, platelet aggregation inhibitors and the like. Commercially available agents include, but are not limited to, Warfarin (3-(1-phenyl-3-oxobuthyl)-4-hydroxycumarine), Antithrombin III or heparin co-factor, sodium dalteparin (sodium salt of de-polymerised heparin), heparan sulfate, sodium enoxaparin, sodium heparin, calcium nadroparin, sodium tinzaparin, monoclonals such as Abciximab; Clopidogrelum, Dipyridamol, Eptifibatid, Ilopros, Ticlopidinum, Tirofiban; enzymatic agents such as Alteplase, Reteplase, Streptodornase, Streptokinase, Urokinase; and others, such as Desirudin. Among these, heparin and heparinoid agents are preferred.

The compositions of the present invention may additionally contain other adjunct components conventionally found in pharmaceutical compositions, at their art-established usage levels. Thus, for example, the compositions may contain additional, compatible, pharmaceutically-active materials such as, for example, antipruritics, astringents, local anaesthetics or anti-inflammatory agents, or may contain additional materials useful in physically formulating various dosage forms of the compositions of the present invention, such as dyes, flavouring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavourings and/or aromatic substances and the like which do not deleteriously interact with the nucleic acid(s) of the formulation.

The agents disclosed herein may be administered by any route, including intradermally, subcutaneously, orally, intraarterially or intravenously. Preferably, administration will be by the intravenous route. Preferably parenteral administration may be provided in a bolus or by infusion. Most preferably the agents or composition is administrated locally, to the site of the infarct and the neighbouring tissue.

The concentration of a disclosed compound in a pharmaceutically acceptable mixture will vary depending on several factors, including the dosage of the compound to be administered, the pharmacokinetic characteristics of the compound(s) employed, the age, sex and condition of the patient, as well as the route of administration. Effective amounts of agents for treating secondary coronary ischemia would broadly range between about 0.01 μg to about 100 mg per kg of body weight, preferably about 0.1 μg to about 10 mg, and most preferably about 1 μg to about 1 mg per kg of body weight of a recipient mammal. The agent may be administered in a single dose or in repeated doses.

The disclosed compound may be administered at any time before, during, or after the onset of primary ischemia. In certain preferred embodiments, the agent is administered after the onset of primary ischemia, but at a time early enough to prevent secondary damage to neighbouring tissue. Preferably said effective amount of an NF-κB p65 inhibitor is administered locally to the organ or tissue within 12 hours, preferably within 8 hours, more preferably within 6 hours, and most preferably within 2 hours from the diagnosis or suspicion of an ischemic condition, such as myocardial infarction or stroke.

According to another embodiment, said effective amount of an NF-κB inhibitor is administered locally to the ischemic area within 2 hours, preferably within 1 hour, more preferably within 0.5 hours, and most preferably at the time of reperfusion of the organ following removal of an obstruction in an occluded artery, e.g. after balloon angioplasty in a cardiac or cerebral artery.

In one embodiment, the inhibitor of NF-κB p65 subunit activation can be administered prior to the onset of ischemia. The onset of ischemia can be predicted in the case of certain medical procedures, such as surgical procedures, transplantation etc. Human organs or tissue need to be preserved for a time in the interval of a few hours up to a few days after having been removed from a donor. The preservation or storage is necessary to give sufficient time for transport, for preparing the intended receiver, and for performing histocompatibility testing of donor and recipient. The mechanisms of injuries sustained during removal of donor organ or tissue, pre-preservation, cold ischemic preservation and reperfusion are believed to be complex and not fully understood. There are various cold storage solutions developed for the preservation of organs and biological tissues prior to implantation, such as electrolyte solutions with suitable additives. According to one embodiment, the inhibitor of NF-κB p65 subunit activation is added to such solution before or during storage of an organ or tissue therein. Preferably the addition of the inhibitor is done shortly before implantation, about 1 to 4 hours before inplantation, most preferably not more than 2 hours before inplantation.

The following examples are intended to further illustrate certain preferred embodiments of the invention and are not limiting in nature. If sources are not specifically described, materials are known and commercially available.

### EXAMPLES

### Example 1

### Creation of Antisense NF-κB p65 Oligonucleotide

The NF-kB(p65) antisense oligonucleotide was purchased from Eurogentec Ltd., (Liège, Belgium) in HPLC purified form, and received as desalted powder. The oligonucleotide was reconstituted in water to the desired concentration.

### Example 2

### Creation of NF-κB Scrambled Oligonucleotide

The sequence of the scrambled NF-kB(p65) antisense oligonucleotide was performed by randomising the arrangement of the base-pairs of the NF-kB(p65) antisense oligonucleotide. The scrambled NF-kB(p65) antisense oligonucleotide sequence was then later checked to ensure no potential homology to known sequences by using the public available data-base search and alignment tool “BLAST” (NCBI).

The scrambled NF-kB(p65) antisense oligonucleotide was purchased from Eurogentec Ltd (Liège, Belgium) in HPLC purified form, and received as desalted powder. The oligonucleotide was reconstituted in water to the desired concentration.

### Example 3

### Myocardial Infarction Mouse Model

The myocardial infarction mouse model used is based on a previously described mouse model, see Michael et al., Myocardial ischemia and reperfusion: a murine model, Am J Physiol. 1995 December; 269(6 Pt 2): H2147-54, and Nossuli et al., A chronic mouse model of myocardial ischemia-reperfusion: essential in cytokine studies, Am J Physiol Heart Circ Physiol., 2000 April; 278(4):H1049-55. This model is recognised as a reliable and repeatable model for studying myocardial infarction in mammals.

Each mouse (All female, Strain C57BI/6g, age 6-7 weeks, weight approx 15 g) was anaesthetised with an intraperitoneal injection of 5-6 μl CRC cocktail (1 part Hypnorm: 1 part Dormicum: 2 parts distilled water) per gram body weight. When fully anaesthetised, the mouse was placed in a supine position with paws taped onto an 11×16 Plexiglas® tile. Their neck was extended by looping the incisors with a thread and taping the thread tautly to the tile, aiding oral-tracheal intubation.

The skin was swabbed with 70% alcohol before a midline skin incision was made from the xiphoid process to the chin. The submandibular glands were retracted to either side with double-hooked retractors, exposing the trachea for visualization of the intubation process. Intubation tubes were 22 gauge intravenous catheters (OPTIVA, Johnson & Johnson) modified to have a shortened and blunted needle. As the tongue was gently held upward with forceps, the catheter was slowly inserted in the trachea.

With the catheter in place, the needle was removed and the catheter connected to a rodent mini-ventilator (Harvard Apparatus, March, Germany) with a 94-98% oxygen air supply (Millennium, Marietta, Ga., USA). The ventilator was set at positive end-expiratory pressure (PEEP) mode of ventilation with a stroke volume of 200 μl and at 200 strokes per minute. When regular chest expansions were observed the catheter was taped in place.

Using double-hooked retractors, the left pectoralis major muscle was retracted to the right and the left rectus thoracis and serratus anterior muscle were retracted to the left, exposing the ribs. A left anterior thoractomy was made laterally at the third intercostal space by microscissor dissection at the upper border of the fourth rib and approximately 3 mm from the sternum, exposing the heart. Care was taken to avoid damaging the intercostal arteries and nerves.

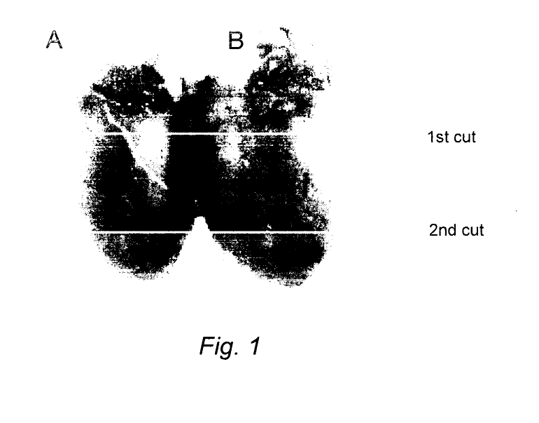

The third and fourth ribs were retracted with micro-dissecting retractors, exposing the left side of the heart. The fat pad was grasped with a pair of forceps and the heart manipulated until the left anterior descending (LAD) coronary artery was visible. At 8× magnification the LAD appeared as an orange-pinkish vessel and was the only coronary artery visible, see FIG. 1, heart A. The LAD was then ligated with 9/0 Ethilon suture (Johnson & Johnson, Brussels, Belgium) approximately 1-2 mm distal to the atrial appendage. This resulted in an immediate pallor in the left ventricle. The tested compounds were administered into the left ventricle (LV) wall at the apical region of the heart immediately after LAD ligation. After 30 minutes, the ligting suture was cut.

The pallor is a visual indication of the induction of ischemia, which occurs in the bottom half of the heart following LAD ligation. While human LAD has some variation in position, the degree of variation in inbred rodent strains is much less significant. Because of the degree of similarity, rodent acute myocardial infarction models are particularly attractive as the region undergoing ischemia following LAD ligation is consistent and reproducible.

The thoracic cavity was closed with 5/0 Chromic catgut absorbable suture (Johnson & Johnson). The retracted muscles were returned to their original positions and the skin was closed with 6/0 Prolene® suture (Johnson & Johnson). The mouse was removed from the Plexiglas® tile and placed in a lateral position, then extubated. If regular breathing did not resume within one minute the mouse was returned to the ventilator. Post-operatively, the mouse was kept in a 31-32° C. incubator to recover. Generally, mice recovered within 3 to 4 hours and resumed normal patterns of drinking, feeding, and mobility.

### Example 4

### Administration of Antisense Compounds to Ischemic Mouse Models

Mice were randomly assigned into three groups. One group consisted of 7 mice and served as a control, receiving only saline. A second group consisted of 10 mice and also served as a control, receiving the scrambled oligonucleotide. Finally, the test group consisted of 10 mice, which received the antisense oligonucleotide. Compounds were administered intra-myocardially. Using an insulin syringe, 20 μl of the appropriate compound was administered into the left ventricle (LV) wall at the apical region of the heart. Mice in the first test group received 20 μl of 1 μM antisense oligonucleotide. Mice in the second test group received 20 μl of 1 μM scrambled oligonucleotide. Control group mice received 20 μl saline. In all cases, the injection occurred immediately after LAD ligation.

The injection was carefully made to prevent the needle from penetrating the LV wall. Such a penetration would cause massive bleeding or hemothorax. Regional cyanosis of the LV wall was evident if the saline or cell suspension was properly injected intramuscularly into the LV wall. An alternate dosing regimen could inject the compounds intravenously in the tail vein. If such a dosing regime is used it may be best accomplished by first dilating the blood vessel, for example by exposing the tail to a heat lamp for approximately one minute.

### Example 5

### Heart Isolation and Removal

Following induction of ischemia and administration of compound, the mice were observed for 20 days. At 20 days post-operative, each mouse was euthanised with an overdose of CRC cocktail. Heparin (5 units in 50 μl saline) was dripped into the abdomen to prevent blood from clotting. The chest cavity was exposed by dissecting the ribs laterally and reflecting the ribs upward with bull-dog clamps. The heart was perfused with 50 ml of 20 mM KCl in formalin at 5-6 ml/minute, under pressure. The pressure of 80 mmHg corresponded to mean mouse arterial pressure. Perfusion was accomplished with a 60 cc syringe connected to a transducer and a 25 gauge needle through a 3-way stopcock.

### Example 6

### Histological Analysis of Ischemic Areas

Each heart was cut transversely at two levels, as shown in FIG. 1. In this figure, two individual mouse hearts are shown, indicated as A and B. In “A”, the left anterior descending (LAD) coronary artery can be seen as a light coloured vessel and is the only artery that is visible. Ligation of the LAD results in the induction of ischemia occurring at the bottom half of the heart. It is a well documented clinical observation that the anatomical location of the LAD coronary artery in humans shows some variation in position from one heart to another, however equally well know is the fact that this degree of variation is much less in rodent inbred strains. This fact has made the use of such acute myocardial infarction models particularly attractive as the region that undergoes ischemia following ligation of the LAD coronary artery is very consistent.

Two transverse lines indicate where tissues sections were taken. The lower line (the 2nd cut) marks the position of tissue sections taken from the region that undergoes ischemia following ligation of the LAD coronary artery. The upper line (the 1st cut) indicates the position of tissue sections taken from an area that does not become ischemic upon ligation, but rather demonstrates that area of tissue that undergoes progressive damage as a direct result form the neighbouring ischemic area.

Tissue sections were stained using standard dyes (H&E, Maison Trichrome). Immunohistochemistry was performed for specific antigens, apoptotic nuclei, PCNA, CD31, Pecam, PPAR-γ or in situ hybridisation for specific genes (Results not shown).

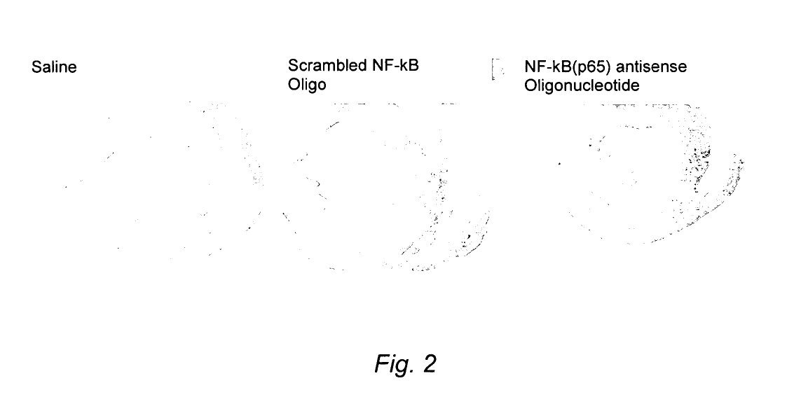

Slides were prepared with tissue sections taken at the first cut position. See FIG. 2, where 2A (saline control), 2B (scrambled oligonucleotide control) and 2C (antisense NF-κB p65 oligonucleotide) show representative slides. In both the saline control (FIG. 2A) and scrambled oligonucleotide (FIG. 2B), there was a significant thinning of the heart muscle wall and an enlargement of the cavity. In contrast, the section from a mouse receiving antisense NF-κB p65 oligonucleotide (FIG. 2C) shows significantly greater integrity of the heart muscle. The cavity is also reduced. The animals receiving the antisense NF-κB p65 oligonucleotide therefore are likely to have improved heart function as a result of decreased degree of secondary tissue damage when compared to animals is the other two test groups. FIG. 2C, when viewed in comparison to FIGS. 2A and 2B demonstrates the significant protective effect conferred by administration of the antisense NF-κB p65 oligonucleotide according to the present invention.

Tissues collected from the second cut area show evidence of ischemia as a result of LAD ligation. This ischemia or myocardial infarct is known to occur following an ischemic event such as LAD ligation. Certain compounds and methods attempt to address the degree of ischemia in this area.

The tissue observed at the first cut location represents an area that does not become ischemic upon ligation. Rather, when untreated, the tissue undergoes progressive secondary damage as a result from the neighbouring ischemic area. It has surprisingly been found, as evidenced by the data described below, that the longer-term, secondary damage can be prevented or reduced through administration of the antisense oligonucleotide.

Pressure and conductance in the left ventricle was measured using a 1.4 French conductance-micromanometer.

Measurements were taken of the LC cavity circumference, infarct size and LV free wall thickness using Leica IM50 computational software. Absolute and relative infarct sizes were statistically evaluated.

### Example 7

### Statistical Analysis of Ischemic Areas

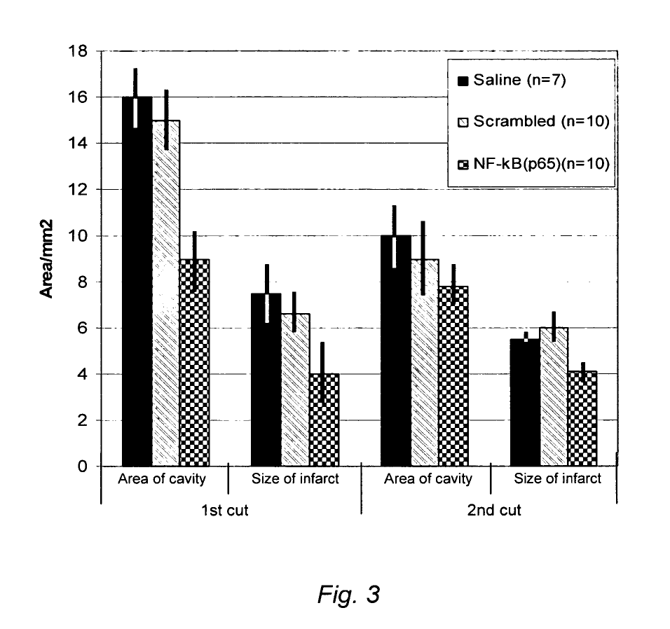

Data from the hearts isolated from experimental models was collected and tabulated. FIG. 3 shows the collective data from the three test groups. As noted above, each mouse underwent LAD ligation followed by immediate intramyocardial administration of 20 μl at 1 μM of scrambled oligonucleotide, antisense NF-κB p65 oligonucleotide, or 20 μl saline. The mice then were observed but otherwise untreated for 20 days following the procedure, when collection and analysis of the cardiac tissues occurred.

As stated, the second cut region is located in the portion of the heart that undergoes ischemia following ligation of LAD. Sections from the second cut region indicate that both the saline control and the scrambled oligonucleotide control had areas of infarct and cavities somewhat larger than those animals, which received antisense NF-κB p65 oligonucleotide as given by SEQ ID NO. 1 (infarcts of 6 and 5.5 versus 4mm², and cavity size of 9 and 10 versus 7.5 mm², see FIG. 3). However, sections taken at the first cut region surprisingly demonstrated a significant reduction in the size of the infarct area in neighbouring tissue as well as a reduced cavity volume in the animals receiving the antisense NF-κB p65 oligonucleotide (infarct of 4 instead of 6.5 or 7.5 mm², cavity size of 9 instead of 15 or 16 mm², see FIG. 3). This indicates that intramyocardial administration of antisense NF-κB p65 subunit oligonucleotide (SEQ ID NO. 1) following LAD ligation has limited effect on the initial ischemic area (second cut area) but it had a very significant positive effect on limiting secondary damage to neighbouring tissue (first cut region). Hence, it provides protection against secondary tissue damage once an infarction has occurred.

### Example 8

### Determination of Inhibitory Effects in Vitro of siRNA Compound IDX0131 on the p65 Subunit of NF-kappaB in HeLa Cells (Experiment Performed During the Priority Year)

It was deemed important to demonstrate that compound IDX 0131 could work also in another cell system. For this purpose, HeLa cells where chosen.

Culture Conditions for HeLa Cells and Transfection Using Cationic Lipids

The human cervix adenocarcinoma cell line HeLa was cultured in Dulbecco's modified Eagle's medium (Invitrogen), supplemented with 10% fetal bovine serum (Invitrogen), 100 ug/ml streptomycin and 100 IU/ml penicillin. The cells were transfected with suitable amount of human Kappaproct® (Index Pharmaceuticals AB, Solna, Sweden), mouse scrambled Kappaproct® or siRNA IDX-0131, (see figure text). by using Gene silencer transfection agent (GTS, CA), according to the manufacturer's protocol.

Cell Harvesting, Sample Preparation and Storage Procedures

To prepare whole cell extract (WCE), cells were harvested with a cell scraper. The cells were collected by centrifugation, washed one time with cold PBS and lysed for 20 min on ice in RIPA lysis buffer (50 mM Tris-HCL, pH 7.5, 150 mM NaCl, 1% NP-40, 0.5% sodium deoxycholate, 0.1% SDS) supplemented with 1 mM DTT and a protease inhibitor cocktail (“Complete-Mini,” Roche). Cell lysates were cleared by centrifugation for 15 min at 4° C. and stored at -70° C. in order to be used. Since not all of the macrophages were attached to the plastic also the “suspension” cells were pelleted and pooled with the attached cells.

Western Analysis of p65

Total protein concentration of the WCE was determined by Bio-Rad protein assay (Bradford). The extracts were analyzed on a 9% SDS-PAGE and transferred to a nitrocellulose membrane. To visualize total amount of protein loaded on the gel, the membrane was stained with Ponceau S solution (Sigma). The membrane was then blocked for 1 hr at room temperature in 5% nonfat milk in PBS, prior to incubation with the p65 antibody (Santa Cruz, Biotechnology, sc-372), diluted 1:1000 in 5% nonfat milk in PBS, at 4° C. over night. After three washes with PBS containing 0.05% Tween-20, a 1:1000 dilution of anti-rabbit IgG conjugated to horseradish peroxidase (DAKO, cat# P0217) in 5% nonfat milk/PBS was used as a secondary antibody. Subsequently, the filters were washed with PBS/0.05% Tween-20, and p65 was visualized by using enhanced chemiluminescence (Amersham Bioscience) according to the manufacturer's recommendations.

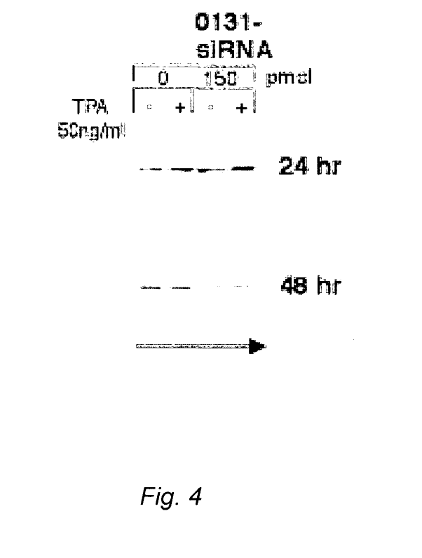

The results are evident from FIG. 4 showing an immuno blot where reduced protein levels of p65 were observed in cells harvested 48 hr after transfection indicating that compound IDX 0131 was also effective in HeLa cells. The results support the claim to utility in other tissues than shown in the mouse heart muscle study.

The foregoing disclosure has been set forth merely to illustrate the invention and is not intended to be limiting. Since modifications of the disclosed embodiments incorporating the spirit and substance of the invention may occur to persons skilled in the art, the invention should be construed to include everything within the scope of the appended claims and equivalents thereof. Patent applications, patents and literature references cited herein indicate the knowledge in this field and are hereby incorporated by reference in their entirety. Where inconsistent interpretations are possible, the disclosure herein controls.

1. A method of preventing, reducing the extent and/or the severity of secondary ischemic damage in a mammalian organ or tissue, comprising a step of administering an effective amount of an NF-κB p65 inhibitor to said organ or tissue.

2. The method according to claim 1, wherein said NF-κB p65 inhibitor is selected from the group consisting of: an antisense NF-κB p65 subunit oligonucleotide; a dominant-negative form of the NF-κB p65 subunit; a decoy; ribosome inhibitors; enzymatic RNA against NF-κB p65; and siRNA constructs.

3. The method according to claim 1, wherein said mammalian organ or tissue is selected from the group consisting of heart, lungs or lung, kidney, liver, brain, skin, and blood vessels.

4. The method according to claim 3, wherein said organ or tissue is a heart or a section thereof.

5. The method according to claim 3, wherein said organ or tissue is a brain or a section thereof.

6. The method according to claim 3, wherein the secondary ischemic damage occurs as the result of reperfusion of an organ or tissue following transplantation of said organ or tissue.

7. The method according to claim 3, wherein the secondary ischemic damage occurs as the result of reperfusion of an organ or tissue following surgical intervention on said organ or tissue or adjacent tissue.

8. The method according to claim 3, wherein the secondary ischemic damage occurs as the result of reperfusion of the heart following removal of an obstruction in a coronary artery.

9. The method according to claim 1, wherein said NF-κB p65 inhibitor is an antisense compound from 8 to 40 nucleotides in length, targeted to a nucleic acid molecule encoding NF-κB p65 subunit or to a fragment or analogue thereof.

10. The method according to claim 1, wherein the antisense NF-κB p65 subunit oligonucleotide is chosen from the group consisting of SEQ ID NO. 1, SEQ ID NO. 2, and functionally equivalent homologues thereof.

11. The method according to claim 1, wherein the antisense NF-κB p65 subunit oligonucleotide is SEQ ID NO. 2.

12. The method according to claim 1, wherein the amount of antisense NF-κB p65 subunit oligonucleotide administered to the mammal is about 100 μg to about 5 mg per kg body weight.

13. The method according to claim 1, wherein the ischemia is the result of vascular occlusion of a cardiac vessel.

14. The method according to claim 1, wherein the ischemia is the result of vascular occlusion of a cerebral vessel.

15. The method according to claim 1, wherein the ischemia is the result of angioplasty.

16. The method according to claim 1, further comprising the step of administering at least one second agent selected from the group consisting of anticoagulants and antithrombotics, vitamin K-antagonists, heparin and heparinoid agents, and platelet aggregation inhibitors.

17. A method of preventing, reducing the extent and/or the severity of secondary myocardial infarct in a mammalian heart, comprising a step of administering an effective amount of an NF-κB p65 inhibitor to said heart.

18. A method of protecting tissue adjacent to area of myocardial infarct in a mammal comprising the step of administering an effective amount of an NF-κB p65 inhibitor locally to the heart of said mammal.

19. The method according to claim 17 or 18, wherein said NF-κB p65 inhibitor is selected from the group consisting of: an antisense NF-κB p65 subunit oligonucleotide; a dominant-negative form of the NF-κB p65 subunit; a decoy; ribosome inhibitors; enzymatic RNA against NF-κB p65; and siRNA constructs.

20. The method according to claim 17 or 18, wherein said effective amount of an NF-κB p65 inhibitor is administered locally to the heart.

21. The method according to claim 17 or 18, wherein said effective amount of an NF-κB p65 inhibitor is administered locally to the heart within 12 hours, preferably within 8 hours, more preferably within 6 hours, and most preferably within 2 hours from the diagnosis of myocardial infarction.

22. The method according to claim 17 or 18, wherein said effective amount of an NF-κB p65 inhibitor is administered locally to the heart within 2 hours, preferably within 1 hour, more preferably within 0.5 hours from the reperfusion of the heart following removal of an obstruction in a coronary artery.

23. The method according to claim 17 or 18, wherein said effective amount of an NF-κB p65 inhibitor is administered locally to the heart substantially at the time of reperfusion of the heart following removal of an obstruction in a coronary artery.

24. The method according to claims 17-18, wherein the antisense NF-κB p65 subunit oligonucleotide is chosen selected from the group consisting of SEQ ID NO. 1, SEQ ID NO. 2, and functionally equivalent homologues thereof.

25. The method according to claims 17-18, wherein the antisense NF-κB p65 subunit oligonucleotide is SEQ ID NO. 2.

26. The method according to claims 17-18, wherein the amount of antisense NF-κB p65 subunit oligonucleotide administered to the mammal is about 100 μg to about 5 mg per g/kg body weight.

27. The method according to claims 17-18, wherein the ischemia is the result of vascular occlusion of a cardiac vessel.

28. The method according to claims 17-18, wherein the ischemia is the result of angioplasty.

29. The method according to claims 17-18, further comprising the step of administering at least one second agent selected from the group consisting of anticoagulants and anti thrombotics, such as vitamin K-antagonists, heparin and heparinoid agents, platelet aggregation inhibitors and the like.

30. The method according to claims 17-18, wherein said NF-κB p65 inhibitor is a siRNA construct.

31. The method according to claim 30, wherein the siRNA construct is directed towards one of the target sequences given in Table 1.

32-46. (canceled)
